(19) United States
(12) Patent Application Publication
(10) Pub. No.: US 2005/0065062 A1
(43) Pub. Date: Mar. 24, 2005

(54) Method of formulating a pharmaceutical composition

(75) Inventors: Stephen B. Roscoe, St. Paul, MN (US); Neal A. Rakow, Woodbury, MN (US); Michael L. Husberg, West St. Paul, MN (US); Lester H. McIntosh III, Green Lane, PA (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/669,390

(22) Filed: Sep. 24, 2003

(51) Int. Cl. A61K 31/00; G06F 19/00; G01N 33/48; G01N 33/50

(52) U.S. Cl. 514/1; 702/19

(57) ABSTRACT

A method of formulating a pharmaceutical composition utilizes at least one model compound as a substitute for at least one pharmaceutical.

### BACKGROUND

Formulation of pharmaceutical compositions for applications involving diffusion through a membrane such as, for example, transdermal pharmaceutical delivery typically involves selection of one or more excipients that are combined with an active pharmaceutical agent (i.e., pharmaceutical). The overall process generally involves repeated preparation, evaluation, and identification of one or more potentially useful formulations that, for example, may be subjected to clinical evaluation.

In some cases, difficulties arise in completing the screening process using the pharmaceutical itself such as, for example, those cases in which the pharmaceutical is rare, expensive, toxic, and/or subject to regulatory restrictions.

It would therefore be desirable to have methods for formulating and evaluating pharmaceutical compositions that reduce the amount of pharmaceutical needed to complete the primary screening process.

### SUMMARY

In one aspect, the present invention provides a method of formulating a pharmaceutical composition comprising:
comparing parameters of at least one pharmaceutical and a plurality of compounds, wherein the parameters comprise at least log(P) and molecular weight;
choosing at least one model compound from the plurality of compounds for each pharmaceutical;
providing at least one model compound-excipient formulation comprising at least one model compound and at least one excipient;
measuring the diffusion of a model compound of at least one model compound-excipient formulation across at least one membrane;
choosing a model compound-excipient formulation based on the measured model compound diffusion; and
combining components comprising the at least one pharmaceutical and the excipient package of the chosen model compound-excipient formulation.

According to the present invention, model compounds can be used in place of pharmaceuticals during formulation and evaluation processes, thereby reducing the amount of the pharmaceutical that is necessary.

### DETAILED DESCRIPTION

As used herein, the term “pharmaceutical” refers to any compound that has at least one therapeutic, disease preventive, diagnostic, or prophylactic effect when administered to an animal and/or a human. Useful pharmaceuticals include, for example, prescription pharmaceuticals, over-the-counter pharmaceuticals, nutriceuticals, vitamins, cosmoceuticals, and pharmaceuticals in development and/or clinical trials. Thus, any pharmaceutical intended for use in animals (e.g., mammals) and/or humans may be screened and/or formulated for delivery across a membrane according to the present invention.

Examples of pharmaceuticals that may be used in practice of the present invention include, but are not limited to, cardiovascular pharmaceuticals (e.g., amlodipine besylate, nitroglycerin, nifedipine, losartan potassium, irbesartan, diltiazem hydrochloride, clopidogrel bisulfate, digoxin, abciximab, furosemide, amiodarone hydrochloride, beraprost, theophylline, pirbuterol, salmeterol, isoproterenol, and tocopheryl nicotinate); anti-infective components (e.g., amoxicillin, clavulanate potassium, itraconazole, acyclovir, fluconazole, terbinafine hydrochloride, erythromycin ethylsuccinate, acetyl sulfisoxazole, penicillin V, cephalexin, erythromycin, azithromycin, tetracycline, ciproflaxin, gentamycin, sulfathiazole, nitrofurantoin, norfloxacin, flumequine, and ibafloxacin, metronidazole, nystatin; psychotherapeutic components (e.g., sertraline hydrochloride, venlafaxine, bupropion hydrochloride, olanzapine, buspirone hydrochloride, alprazolam, methylphenidate hydrochloride, fluvoxamine maleate, and ergoloid mesylates); gastrointestinal products (e.g., lansoprazole, ranitidine hydrochloride, famotidine, ondansetron hydrochloride, granisetron hydrochloride, sulfasalazine, and infliximab); respiratory therapies (e.g. loratadine, fexofenadine hydrochloride, cetirizine hydrochloride, fluticasone propionate, salmeterol xinafoate, and budesonide); antihistamines (e.g., diphenhydramine, chlorpheniramine, terfenadine); cholesterol reducers (e.g., atorvastatin calcium, lovastatin, bezafibrate, ciprofibrate, and gemfibrozil); cancer and cancer-related therapies (e.g., paclitaxel, carboplatin, tamoxifen citrate, docetaxel, epirubicin hydrochloride, leuprolide acetate, bicalutamide, goserelin acetate implant, irinotecan hydrochloride, gemcitabine hydrochloride, and sargramostim); blood modifiers (e.g., epoetin alfa, enoxaparin sodium, and antihemophilic factor); antiarthritic components (e.g., celecoxib, nabumetone, misoprostol, and rofecoxib); AIDS and AIDS-related pharmaceuticals (e.g., lamivudine, indinavir sulfate, and stavudine); diabetes and diabetes-related therapies (e.g., metformin hydrochloride, insulin, troglitazone, and acarbose); biologicals (e.g., hepatitis B vaccine, and hepatitis A vaccine); immune response modifiers (e.g., purine derivatives, adenine derivatives, and CpGs); hormones (e.g., estradiol, mycophenolate mofetil, and methylprednisolone); enzyme inhibitors (e.g., zileuton, captopril, and lisinopril); antihypertensives (e.g., propranolol); leukotriene antagonists; anti-ulceratives such as H2 antagonists; antinauseants (e.g., scopolomine); anticonvulsants (e.g., carbamazine); immunosuppressives (e.g., cyclosporine); analgesics (e.g., tramadol hydrochloride, fentanyl, metamizole, ketoprofen, morphine sulfate, lysine acetylsalicylate, acetaminophen, ketorolac tromethamine, morphine, loxoprofen sodium, and ibuprofen); dermatological products (e.g., isotretinoin and clindamycin phosphate); anesthetics (e.g., propofol, midazolam hydrochloride, and lidocaine hydrochloride); migraine therapies (e.g., ergotamine, melatonin, sumatriptan, zolmitriptan, and rizatriptan); sedatives and hypnotics (e.g., zolpidem, zolpidem tartrate, triazolam, and hycosine butylbromide); imaging components (e.g., iohexol, technetium, TC99M, sestamibi, iomeprol, gadodiamide, ioversol, and iopromide); anti-inflammatory therapies (e.g. hydrocortisone, prednisolone, triamcinolone, naproxen, and piroxicam); local anesthetics (e.g., benzocaine, propofol); antitussives (e.g., codeine, dextromethorphan);sedatives (e.g., phenobarbital); anticoagulants (e.g., heparin); antiarrhythmic agents (e.g., flecainide); antiemetics (e.g., metaclopromide, ondansetron); anti-obesity agents; diagnostic and contrast components (e.g., alsactide, americium, betazole, histamine, mannitol, metyrapone, petagastrin, phentolamine, radioactive B₁₂, gadodiamide, gadopentetic acid, gadoteridol, perflubron, cyclosporine, sildenafil citrate, paclitaxel, ritonavir, and saquinavir); pharmaceutically acceptable salts and esters thereof; and combinations thereof. Further examples of suitable pharmaceuticals are listed in the “PDR electronic library on CD-ROM”, Medical Economics Library, Montvale, N.J. (2003).

Once a pharmaceutical of interest is chosen, physical parameters relating to that pharmaceutical are obtained, for example, by direct experimentation, calculation, or by consulting published data. At least two physical parameters should be obtained for the pharmaceutical including: (1) the octanol/water partition coefficient (i.e., log(P)), and (2) the molecular weight. These two parameters are generally useful for describing the hydrophilic/lipophilic balance and molecular size of the pharmaceutical, both properties typically being important in membrane diffusion processes. Optionally, additional parameters may be obtained including, for example, the number of freely rotatable bonds and/or the number of H-bond donors and acceptors. These latter parameters may further may be useful to refine the selection of a model compound for the pharmaceutical, but typically have less effect on membrane diffusion than log(P)) and molecular weight.

Methods for experimentally determining log(P) are well known, and are described for example in ASTM El 147-92 (1997) “Standard Test Method for Partition Coefficient (n-OctanoVWater) Estimation by Liquid Chromatography”, the disclosure of which is incorporated herein by reference. This test method describes a procedure for the estimation of log(P) of chemicals over the range from 0 to 8, using an empirically derived equation to relate the octanol/water partition coefficient to an experimentally determined retention time on a liquid chromatographic column.

Another experimental method determining log(P) is described, for example, in Title 40, Chapter 1 of the U.S. Code of Federal Regulations, Jul. 1, 2001 edition, Subpart E, §799.6755 “TSCA Partition Coefficient (n-octanol/water), Shake Flask Method”, pp. 274-277, the disclosure of which is incorporated herein by reference. In this method, a compound to be evaluated is placed in a flask containing n-octanol and water, and then shaken. After allowing the n-octanol and water to separate, the amount of the compound in each of the n-octanol and water is then measured by conventional techniques.

Alternatively, or in addition, log(P) may be calculated using a fragment-correction method as described, for example, by Ghose et al. in “Journal of Computational Chemistry”, 1988, vol. 9, pp. 80-90; or by using commercially available computer software such as, for example, that marketed under the trade designation “CLOG(P)” (e.g., “CLOG(P) 4.0”) by BioByte Corporation, Claremont, Calif.; “LOGKOW/KOWWIN” by Syracuse Research Corporation, Syracuse, N.Y.; “LOG(P) DB” by Advanced Chemistry Development, Toronto, Canada; “CACHELOG(P)” by the CAChe group, Beaverton, Oreg.; and “CSLOG(P)” by ChemSilico, LLC, Tewksbury, Mass. Additionally, log(P) values may be obtained from a wide variety of literature sources such as, for example, C. L. Yaws “Chemical Properties Handbook”, New York: McGraw-Hill, pp. 364-388 (1999), and the “Handbook of Physical Properties of Organic Chemicals”, edited by Philip H. Howard and William M. Meylan; Boca Raton: Lewis Publishers (1997).

Molecular weight can be readily obtained by well-known methods (e.g., inspection of the molecular formula or freezing point depression). The number of freely rotatable bonds and the number of H-bond donors and acceptors may also be readily obtained by examination of the structural formula of the compound. Further details concerning methods for determining the number of freely rotatable bonds of compounds are described, for example, by Veber et al. in “Journal of Medicinal Chemistry” (2002), vol. 45, pp. 2615-2623, and the number of H-bond donors and acceptors as described in, for example, Lipinski et al. in “Experimental and Computational Approaches to Estimate Solubility and Permeability in Pharmaceutical Discovery and Development Settings”, Advanced Drug Delivery Reviews (1997), vol. 23(1-3), pp. 3-25.

Calculation of parameters of compounds (including, e.g., pharmaceuticals and dyes) may be particularly useful, for example, if synthesis of a particular compound is required in order to physically measure the parameters.

Compounds that may be used as model compounds include any known or predicted compounds. Typically, useful model compounds are organic compounds. Compounds may be obtained, for example, by synthesis according to known methods or from a commercial supplier such as, for example, Aldrich Chemical Company, Milwaukee, Wis.

One particularly useful class of compounds that can be used as model compounds according to the present invention includes dyes (including leuco dyes). The spectral properties of dyes facilitate measurement of their concentration (e.g., in absolute and/or relative terms) in solution using techniques such as, for example, an aided or unaided human eye, fluorescence spectroscopy, absorption spectroscopy, colorimetry, and reflectance spectroscopy. Published compilations of dyes and their commercial sources include, for example, “The Colour Index International”, 3rd Edition, and revisions; published by The Society of Dyers and Colourists, Bradford, West Yorkshire, England (1971 to present). Also, numerous methods for synthesizing dyes are known and include those described, for example, in “Color Chemistry: Syntheses, Properties and Applications of Organic Dyes and Pigments”, edited by A. T. Peters and H. S. Freeman, New York: Elsevier Applied Science (1991). Representative classes of dyes include, for example, xanthene dyes (including thioxanthene dyes), aromatic hydrocarbon dyes (e.g., perylene dyes), imide dyes (including perylene imide dyes and naphthalimide dyes), coumarin dyes, indigoid dyes (including thioindigoid dyes), aniline dyes, methine dyes (including polymethine dyes), azo dyes, cyanine dyes (including hemicyanine dyes), carotinoid dyes, styryl dyes, quinaldine dyes, anthraquinones dyes, nitro dyes, nitroso dyes, azo dyes, diazo dyes, and combinations thereof.

Representative classes of useful leuco dyes include, for example, biphenol leuco dyes, phenolic leuco dyes, indoaniline leuco dyes, acylated azine leuco dyes, phenoxazine leuco dyes, and phenothiazine leuco dyes. Also useful are leuco dyes such as those described, for example, in U.S. Pat. Nos. 3,445,234 (Cescon et al.); 4,021,250 (Sashihara, et al.); 4,022,617 (McGuckin); and 4,368,247 (Fletcher, Jr., et al.). Methods for synthesizing leuco dyes are well known and include those described, for example, in “Chemistry and Applications of Leuco Dyes”, edited by R. Muthyala, New York: Plenum Press (1997).

Once obtained, parameters of the pharmaceutical and a plurality of compounds are compared, and one or more model compounds are typically chosen that have parameters that at least approximate the parameters of the pharmaceutical. Typically, those compounds that most closely approximate the parameters of the pharmaceutical give the best approximation of the pharmaceutical in testing, however latitude in choice of the compound to account for factors such as difficulty in obtaining the compound (e.g., a previously unknown compound) is acceptable. For example, while any value of log(P) may be used, best results are typically obtained if the absolute value of the difference in log(P) between the compound and the pharmaceutical is less than or equal to about 3, 2.5, 2.0, 1.5, 1.0, 0.5, 0.2, or even less than or equal to about 0.1. Similarly, while any value of molecular weight may be used, best results are typically obtained if the absolute value of the difference in molecular weight between the compound and the pharmaceutical is less than or equal to about 150, 100, 75, 50, 40, 30, 20, or even less than or equal to about 10 grams per mole.

Once chosen, the model compound is evaluated for diffusion across a membrane. Suitable membranes include, for example, synthetic polymer membranes (e.g., cellulose acetate sheets, polymeric membranes containing ethyl cellulose, phospholipids, cholesterol, and mineral oil, polyurethane polymers containing poly(ethylene glycol) block segments, synthetic zeolites incorporated into poly(styrene), silicone rubbers, laminated polymer sheets containing alternating hydrophilic and hydrophobic sheets, filter papers or membranes loaded with organic liquids, and cultured cell membranes); hairless mouse skin; snake skin; pig skin; and cadaver skin. Further details concerning suitable synthetic membranes that are useful as substitutes for mammalian skin in permeation testing are described by, for example, Houk et al. in “Membrane Models for Skin Penetration Studies”, Chemical Reviews (1988), vol. 88(3), pp. 455-472, and by Hatanaka et al. in “Prediction of Skin Permeability of Drugs. II. Development of Composite Membrane as a Skin Alternative”, International Journal of Pharmaceutics (1992), vol. 79, pp. 21-28.

Excipients are compounds that serve to assist or retard the diffusion of the pharmaceutical across a membrane. Many excipients are known in the art and include, for example: terpenes (e.g., alpha-terpineol, (+)-terpinen-4-ol, 1,3,3-trimethyl-2-oxabicyclo[2.2.2]octane, p-cymene); alcohols including polyols (e.g., (S)-(+)-2,2-dimethyl-1,3-dioxolane-4-methanol, (R)-(−)-2,2-dimethyl-1,3-dioxolane-4-methanol, 1,2-propanediol, butane-1,3-diol, diethylene glycol monoethyl ether, tetrahydrofurfuryl alcohol polyethylene glycol ether, ethylene glycol, ethanol, propanol, glycerol); esters (e.g., propylene glycol laurate, isopropyl myristate, isopropyl palmitate, ethylhexyl palmitate, butyl dodecanoate, lauric acid lauryl ester, propanoic acid 2-hydroxy-dodecyl ester, linoleic acid butyl ester, lauric acid methyl ester, methyl dodecanoate, dodecyl dodecanoate, lauric acid methyl ester, methyl dodecanoate, lauric acid ethyl ester, ethyl dodecanoate, oleic acid ethyl ester, (−)-methyl L-lactate, ethyl lactate, lauryl lactate, butyl lactate); amides (e.g., N,N-dimethylformamide, N,N-dimethylacetamide, N-laurylpyrrolidone, N-octylpyrrolidone, N-(2-hydroxyethyl)pyrrolidone, N-methylpyrrolidone, 1-dodecylazacycloheptan-2-one and other N-substituted alkylazacycloalkyl-2-ones); halocarbons (e.g., chloroform, methylene chloride); fatty acids (e.g., lauric acid, oleic acid, isostearic acid, linoleic acid, capric acid, neodecanoic acid); cationic, anionic, and nonionic surfactants (e.g., sodium dodecyl sulfate, polyoxamers); anticholinergic agents (e.g., benzilonium bromide, oxyphenonium bromide), oils (e.g., tea tree oil, mineral oil), ketones (e.g., acetone), ethers (e.g., tetrahydrofuran); dimethyl sulfoxide; acetonitrile; aqueous solvents (e.g., water, buffered saline, Lactated Ringer's), and combinations thereof. As used herein, the term “excipient package” collectively refers to the combination of all excipient compounds in the composition being referred to (e.g., a model compound-excipient formulation or a pharmaceutical composition).

Useful commercially available excipients include, for example, those available under the trade designations “LABRASOL” or “LABRAFIL” (e.g., “LABRIFIL M 1944 CS” or “LABRIFIL M 2130 CS”) from Gattefossé Corporation, Paramus, N.J.

Model compound-excipient formulations and pharmaceutical compositions may be prepared by combining one or more excipients with one or more dyes or pharmaceuticals, respectively, using well known mixing and handling techniques.

Diffusion measurements of one or more dyes across a membrane, alone or in combination with at least one excipient, may be determined according to any suitable method(s). Typical methods utilize a Franz cell or similar testing apparatus that has two chambers separated by a membrane. A Franz cell has a membrane (e.g., skin) held between two glass half-cells, typically one glass half-cell contains a test solution or transdermal patch that comprises, for example, a model compound-excipient formulation or a pharmaceutical composition, and the other glass half-cell contains a recipient solution representative of serum. Thus, the model compound-excipient formulation or pharmaceutical composition and recipient composition each contact the membrane, and diffuse through the membrane over time.

Typically, each Franz cell requires about two square centimeters of membrane, and must be emptied and carefully refilled with recipient solution for each diffusion measurement. Typically, diffusion measurements are made in multiples (e.g., quadruplicate) in order to obtain statistically reliable data. Such diffusion measurements are typically laborious, and require considerable operator intervention at each time point (e.g., every six hours) to remove an aliquot of the recipient solution for testing. Each aliquot removed is then typically analyzed, for example, by high performance liquid chromatography (i.e., HPLC).

The amount of model compound that has diffused through the membrane into the recipient solution can be measured by spectroscopic techniques including, for example, reflectance spectroscopy, fluorescence spectroscopy, or absorption spectroscopy, or by other well known techniques such as HPLC, gas chromatography, and the like. If a leuco dye is used, chemical reaction to generate the dye form is typically carried out before measuring the amount of it that is present, for example, using any of the foregoing spectroscopic techniques. Examples of chemical reactions include oxidation, and derivatization.

Some measurement techniques do not require removal of an aliquot to determine the concentration of model compound in the recipient solution. For example, if the model compound is a dye, measurement data may be collected and analyzed essentially simultaneously, or it may be collected in real time, for example, using optical techniques such as an optical scanner or camera (e.g., a CCD camera) and recorded as an image that can be analyzed later by computational or spectrophotometric methods (e.g., reflectance spectrophotometry). Accordingly, such techniques may be used to simultaneously measure dye diffusion in a plurality of diffusion cells, for example, by using a measurement apparatus of the type described in commonly assigned U.S. Patent application entitled “APPARATUS AND METHOD FOR MEASURING MEMBRANE DIFFUSION”, bearing Attorney Case No. 58917US002, filed concurrently herewith, the disclosure of which is incorporated herein by reference. Other exemplary useful measurement apparatus may be found in for, example, U.S. Patent Application Publication No. 2002/0025509 (Cima et al.).

Franz cells are commercially available, for example, from the Crown Glass Company, Somerville, N.J. and from PermeGear, Bethlehem, Pa. Methods for using Franz cells are well known and are described, for example, in U.S. Pat. No. 4,751,087 (Wick). As typically used, one or more pharmaceuticals, typically in combination with one or more excipients, is placed onto a stretched membrane of the Franz cell and the model compound is allowed to diffuse through the membrane followed by assay (e.g., by high performance liquid chromatography or microbial challenge).

Pharmaceutical compositions and model compound-excipient formulations may optionally include various ingredients commonly used with transdermal compositions, such as, for example, antioxidants and preservatives, coloring and diluting agents, emulsifying and suspending agents, ointment bases, thickeners, fragrances, and combinations thereof.

Pharmaceutical compositions and model compound-excipient formulations can be applied to the membrane and/or skin of a live mammal in any suitable form (e.g., in the form of a liquid; a viscid aqueous solution such as a mucilage or jelly; an emulsion, including an oil-in-water emulsion and a water-in-oil emulsion; or a suspension such as a gel, lotion, or mixture). Suitable forms are well known in the art and are described, for example, by J. G. Naim, in “Remington's Pharmaceutical Sciences”, 17th edition, A. F. Gennaro, ed., Mack Publishing Company: Easton, Pa., pp. 1492-1517 (1985).

Model compound-excipient formulations and pharmaceutical compositions used in practice of the present invention may be included in a transdermal delivery device (e.g., a transdermal adhesive patch), such as those described, for example, in U.S. Pat. Nos. 3,598,122 (Zaffaroni); 3,598,123 (Zaffaroni); 3,731,683 (Zaffaroni); 3,797,494 (Zaffaroni); 4,435,180 (Leeper); 5,814,599 (Mitragotri et al.); or 5,879,322 (Lattin et al.).

Transdermal drug delivery devices typically involve a carrier (such as a liquid, gel, or solid matrix, or a pressure-sensitive adhesive) into which a composition (e.g., pharmaceutical) to be delivered is incorporated. Transdermal delivery devices known in the art include, for example, reservoir type devices involving membranes that control the rate of pharmaceutical and/or excipient delivery to the skin, single layer devices involving a dispersion or solution of drug and excipients in a pressure-sensitive adhesive matrix, and more complex multi-laminate devices involving several distinct layers, e.g., layers for containing drug, for containing skin penetration enhancer, for controlling the rate of release of the drug and/or skin penetration enhancer, and for attaching the device to the skin.

In addition, pharmaceutical compositions and model compound-excipient formulations incorporated into transdermal delivery systems, such as reservoir systems with rate-controlling membranes, including microencapsulation, macroencapsulation, and membrane systems; reservoir systems without rate-controlling membranes (such as hollow fibers, microporous membranes and porous polymeric substrates and foams); monolithic systems including those where the composition is physically dispersed in a nonporous polymeric or elastomeric matrix; and laminated structures including those where the reservoir layer is chemically similar to outer control layers and those where the reservoir layer is chemically dissimilar to outer control layers.

Further details concerning transdermal delivery devices may be found in, for example, U.S. Pat. Nos. 5,494,680 (Peterson) and 6,086,911 (Godbey), and U.S. Patent Application Publication 2003/0072792 (Flanigan et al.), the disclosures of which is incorporated herein by reference.

Once one or more model compound-excipient formulations having the desired membrane diffusion characteristics are chosen, then one or more pharmaceutical compositions are prepared that correspond to those formulations, but with the model compound(s) replaced with the pharmaceutical(s) that they model.

The pharmaceutical compositions may then be subjected to further evaluation (e.g., in vivo clinical testing includes contacting the pharmaceutical composition with the skin of at least one live mammal and observing the results). The model compound-excipient formulations that are chosen may be model compound-excipient formulations wherein the membrane diffusion characteristics were actually tested, or they may be model compound-excipient formulations that fall within or near a range of model compound-excipient formulations that have the desired membrane diffusion characteristics.

The present invention will be more fully understood with reference to the following non-limiting examples in which all parts, percentages, ratios, and so forth, are by weight unless otherwise indicated.

### EXAMPLES

Unless otherwise noted, all reagents used in the examples were obtained, or are available, from general chemical suppliers such as Sigma-Aldrich Corporation, Saint Louis, Mo., or may be synthesized by known methods.

Log(P) values compounds reported in Table 1 were calculated using software marketed under the trade designation “KOWWIN” by Syracuse Research Corporation, Syracuse, N.Y.

Membrane diffusion measurements were carried out using a Franz diffusion cell, obtained from PermeGear, Inc., Bethlehem, Pa.

As used herein,

“tetraglycol” refers to tetrahydrofurfuryl alcohol polyethylene glycol ether; and
“lauroglycol” refers to propylene glycol laurate which was obtained under the trade designation “LAUROGLYCOL FCC” from Gottefosse Corporation, Paramus, New Jersey.

In the following Tables “nm” means not measured.

Table 1 (below) reports log(P) and molecular weight (MW) for a series of dyes.

TABLE 1

| DYE | MW | Log(P) |
|---|---|---|
| Patent Blue VF | 566.68 | −5.34 |
| Eosin B | 624.08 | −2.96 |
| Acriflavine hydrochloride | 259.74 | −2.64 |
| Phenosafranine | 322.8 | −2.45 |
| Brilliant Sulfaflavine | 418.4 | −2.39 |
| Pyrogallol Red | 400.37 | −1.83 |
| Alizarin Red S | 342.26 | −1.78 |
| Nuclear Fast Red | 357.28 | −1.6 |
| Safranine O | 350.85 | −1.35 |
| Sunset Yellow FCF | 452.37 | −1.18 |
| Acid Blue 92 | 695.59 | −1.14 |
| Alphazurine A | 690.82 | −1 |
| Pyronin Y | 302.81 | −0.97 |
| FIAT Brilliant Sulfaflavine FF | 382.39 | −0.83 |
| Methyl Orange | 327.34 | −0.66 |
| Methylene Violet 3RAX | 378.91 | −0.37 |
| Neutral Red | 288.78 | −0.33 |
| Erythrosin B | 879.87 | −0.29 |
| Naphthol Yellow S | 358.2 | −0.26 |
| Alizarin Blue Black B | 610.52 | 0.1 |
| Acridine Yellow G | 273.77 | 0.15 |
| Basic Blue 3 | 359.9 | 0.28 |
| Thioflavin T | 318.87 | 0.33 |
| Acid Yellow 99 | 496.35 | 0.45 |
| Nitro Red | 512.39 | 0.58 |
| Acid Red 4 | 380.36 | 0.64 |
| Direct Yellow 8 | 518.55 | 0.64 |
| Mordant Red 19 | 430.81 | 0.68 |
| Tropaeolin O | 316.27 | 0.69 |
| Thionin | 287.34 | 0.79 |
| Carminic acid | 492.4 | 0.97 |
| Crystal Violet | 407.99 | 0.98 |
| Acid Blue 41 | 487.47 | 0.99 |
| Lacmoid | 213.19 | 1.02 |
| Acid Orange 8 | 364.36 | 1.11 |
| Pinacryptol Yellow | 446.48 | 1.12 |
| Fast Red ITR | 258.34 | 1.19 |
| Quinaldine Red | 430.33 | 1.22 |
| Acridine orange hydrochloride hydrate | 265.36 | 1.24 |
| Dinitroresorcinol | 200.11 | 1.25 |
| Resorcein | 428.39 | 1.47 |
| Morin | 302.24 | 1.48 |
| Chromoxane Cyanine R | 536.4 | 1.49 |
| New Fuschin | 365.91 | 1.54 |
| Gallocyanine | 336.73 | 1.56 |
| Pararosaniline base | 305.38 | 1.63 |
| Alkali Blue 6B | 573.65 | 1.66 |
| Eriochrome Black T | 461.39 | 1.78 |
| Plasmocorinth B | 518.82 | 1.79 |
| Fast Violet B | 256.31 | 1.85 |
| Rhodamine B | 479.02 | 1.85 |
| Lumichrome | 242.24 | 1.86 |
| Acid Yellow 40 | 584.99 | 1.94 |
| Eriochrome Blue Black B | 416.39 | 1.96 |
| Eriochrome Blue Black R | 416.39 | 1.96 |
| Acid Orange 74 | 493.38 | 2.01 |
| o-nitroaniline | 138.13 | 2.02 |
| Disperse Yellow 9 | 274.24 | 2.04 |
| Acid Yellow 34 | 414.81 | 2.04 |
| Victoria Blue R | 458.05 | 2.1 |
| Rhodamine 110 | 366.81 | 2.14 |
| Methylene Violet (Bernthsen) | 256.33 | 2.2 |
| Acid Blue 25 | 416.39 | 2.22 |
| Orange IV | 353.4 | 2.25 |
| Metanil Yellow | 375.38 | 2.25 |
| Pyrocatechol Violet | 386.38 | 2.25 |
| Crocein Orange G | 350.33 | 2.35 |
| Orange II | 350.33 | 2.35 |
| Azure C | 277.78 | 2.38 |
| Methyl Eosin | 683.93 | 2.41 |
| Celestine Blue | 363.8 | 2.51 |
| Acid Alizarin Violet N | 366.33 | 2.57 |
| Acridine Yellow base | 237.3 | 2.58 |
| Lacmoid | 429.39 | 2.63 |
| Congo Red | 696.67 | 2.63 |
| Acid Red 151 | 454.44 | 2.68 |
| Pinacyanoyl chloride | 388.94 | 2.7 |
| Cresyl Violet Acetate | 321.34 | 2.83 |
| Janus Green B | 511.07 | 2.84 |
| Fluorescamine | 278.27 | 2.9 |
| Ethyl Eosin | 714.07 | 2.9 |
| Disperse Blue 1 | 268.28 | 2.98 |
| Auramine O | 303.84 | 2.98 |
| Rosolic Acid | 290.32 | 3.03 |
| Indoine Blue | 506.01 | 3.08 |
| Indigo | 262.27 | 3.11 |
| Mordant Brown 4 | 332.28 | 3.11 |
| Lapachol | 242.28 | 3.13 |
| Disperse Red 19 | 330.35 | 3.14 |
| Disperse Violet 1 | 238.15 | 3.16 |
| Alizarin | 240.21 | 3.16 |
| 4-phenylazoaniline | 197.24 | 3.19 |
| Pararosaniline acetate | 347.42 | 3.19 |
| Mordant Brown 48 | 352.7 | 3.2 |
| Disperse Blue 3 | 296.33 | 3.28 |
| Victoria Blue B | 506.1 | 3.28 |
| Curcumin | 368.39 | 3.29 |
| Fat Brown RR | 262.32 | 3.3 |
| Naphthol AS BI phosphate | 452.21 | 3.34 |
| Fluorescein | 332.31 | 3.35 |
| Nile Blue A | 732.86 | 3.39 |
| Naphthol Blue Black | 616.5 | 3.4 |
| Naphthol AS acetate | 305.34 | 3.47 |
| Azure B | 305.83 | 3.48 |
| fluorescein diacetate | 416.39 | 3.5 |
| 4-(4-nitrophenylazo)catechol | 259.22 | 3.55 |
| 3-nitroalizarin | 285.21 | 3.56 |
| Xylene Cyanole FF | 538.62 | 3.57 |
| Disperse Orange 3 | 242.24 | 3.59 |
| Acridine orange base | 265.36 | 3.76 |
| Aurin Tricarboxylic Acid | 422.35 | 3.8 |
| Methyl Red | 269.31 | 3.83 |
| Sudan Orange G | 214.22 | 3.85 |
| Acid Blue 129 | 458.47 | 3.86 |
| Disperse Yellow 3 | 269.31 | 3.98 |
| Methylene Green | 378.86 | 4.01 |
| Rhodamine 6G | 479.02 | 4.02 |
| 2-Phenylthiochromen-4-one | 238.31 | 4.03 |
| Victoria Pure Blue BO | 514.16 | 4.06 |
| Disperse Orange 11 | 237.27 | 4.07 |
| Cresolphthalein | 346.38 | 4.15 |
| Disperse Red 1 | 314.35 | 4.2 |
| Quinoline Yellow, spirit soluble | 273.29 | 4.21 |
| Indophenol Blue | 276.34 | 4.21 |
| 4-(4-nitrophenylazo)resorcinol | 259.22 | 4.25 |
| Disperse Blue 14 | 266.3 | 4.25 |
| Cresol Purple | 382.43 | 4.3 |
| Cresol Red | 382.43 | 4.3 |
| Nile Red | 318.38 | 4.38 |
| Mordant Brown 24 | 375.3 | 4.42 |
| Naphthol AS | 263.3 | 4.47 |
| Chlorophenol Red | 423.28 | 4.5 |
| Disperse Orange 25 | 323.36 | 4.69 |
| Azure A | 291.8 | 4.72 |
| Malachite Green Carbinol Base | 346.48 | 4.74 |
| Alizarin Yellow GG | 287.23 | 4.76 |
| Mordant Orange 1 | 287.23 | 4.76 |
| Eosin Y | 691.88 | 4.8 |
| Disperse Red 13 | 348.79 | 4.85 |
| Naphthol AS BI | 372.23 | 4.88 |
| Crystal Violet lactone | 415.54 | 4.95 |
| 4-(4-nitrophenylazo)-1-naphthol | 293.28 | 5.2 |
| Sudan Blue | 294.36 | 5.24 |
| Toluidine Blue O | 305.83 | 5.26 |
| Xylenol Blue | 410.49 | 5.4 |
| a-naphtholphthalein | 418.45 | 5.41 |
| Sudan I | 248.29 | 5.51 |
| Disperse Orange 1 | 318.34 | 5.8 |
| Methylene Blue | 373.9 | 5.85 |
| Para Red | 293.28 | 5.9 |
| Eosin B spirit soluble | 580.11 | 5.92 |
| Orange OT | 262.32 | 6.05 |
| Disperse Yellow 7 | 316.37 | 6.3 |
| Naphtholbenzein | 374.44 | 6.4 |
| Toluidine Red | 307.3 | 6.45 |
| Rose Bengal | 1017.64 | 6.58 |
| Sudan II | 276.34 | 6.6 |
| Rhodamine B base | 442.56 | 6.63 |
| Disperse Orange 13 | 352.4 | 6.93 |
| Sudan Blue II | 350.46 | 7.2 |
| Sudan III | 352.4 | 7.63 |
| Sudan Red 7B | 379.47 | 7.93 |
| Erythrosin B spirit soluble | 835.9 | 8.05 |
| Oil Blue N | 378.52 | 8.18 |
| Sudan IV | 380.45 | 8.72 |
| Sudan Red B | 380.45 | 8.72 |
| Sudan Black B | 456.55 | 8.81 |
| Oil Red EGN | 394.48 | 9.27 |
| Oil Red O | 408.51 | 9.81 |

### Example 1

The molecular weight and log(P) values of testosterone (Log(P)=3.3; MW=288.4 grams/mole) were compared with those of the dyes listed in Table 1. Fat Brown RR (Log(P)=3.3; MW=262.3 grams/mole) was selected as a model compound for testosterone based on a comparison of these parameters and commercial availability.

Saturated solutions of Fat Brown RR in each of the excipients alpha-terpineol, tetraglycol, isostearic acid and propylene glycol were prepared (4 solutions) in screw cap vials by combining Fat Brown RR with each excipient in separate vials and agitating the vials overnight at room temperature, then filtering the solutions to remove solid particulates. Saturated solutions of testosterone in each of the excipients alpha-terpineol, tetraglycol, isostearic acid and propylene glycol (4 solutions) were similarly prepared.

For each membrane diffusion measurement, a Franz diffusion cell was assembled using freshly excised hairless mouse skin. The hairless mouse skin was mounted with the epidermal side toward the top (donor) chamber of the Franz cell. The lower (receiver) chamber of the Franz cell was filled with 0.01 molar phosphate buffer having a pH of approximately 6.9 to approximately 7 and having an ionic strength of approximately 0.155. A 2-milliliter portion of the saturated solution to be tested was placed in the top (donor) chamber of the Franz cell. The Franz cell was placed in a constant temperature and constant humidity chamber maintained at 34° C. to 35° C. and about 60 percent relative humidity. As the buffer in the receiver chamber was magnetically stirred, aliquots were removed periodically for analysis by high performance liquid chromatography (in the case of testosterone) or UV-VIS absorption spectroscopy (in the case of Fat Brown RR). After each aliquot was removed, the chamber was refilled with a volume of fresh buffer equal to the volume of the aliquot that was removed.

A comparison of the cumulative amount of Fat Brown RR and testosterone that were delivered across the hairless mouse skin into the buffer in the receiver chamber of the Franz cell as a function of time, expressed as micrograms of compound per milliliter of buffer solution (μg/mL), is reported in Table 2 (below).

TABLE 2

| | | CUMULATIVE AMOUNT OF | | | | |
|---|---|---|---|---|---|---|
| | | COMPOUND DIFFUSED ACROSS | | | | |
| | | SKIN, μg/mL | | | | |
| | | 0 | 6 | 12 | 18 | 24 |
| EXCIPIENT | COMPOUND | hours | hours | hours | hours | hours |
| Tetraglycol | Fat Brown RR | 0 | 13 | 25 | 39 | 52 |
| | Testosterone | 0 | 23 | 46 | 81 | 119 |
| Isostearic acid | Fat Brown RR | 0 | 41 | 92 | 169 | 262 |
| | Testosterone | 0 | 74 | 192 | 418 | 740 |
| Propylene | Fat Brown RR | 0 | 84 | 221 | 445 | 677 |
| glycol | Testosterone | 0 | 79 | 216 | 546 | 956 |
| alpha- | Fat Brown RR | 0 | 168 | 444 | 812 | 1162 |
| Terpineol | Testosterone | 0 | 605 | 1585 | 3043 | 4414 |

The results in Table 2 show that the same relative order (alpha-terpineol>propylene glycol>isostearic acid>tetraglycol) for membrane diffusion rate was obtained using testosterone and Fat Brown RR.

### Example 2

The molecular weight and log(P) values of testosterone (Log(P)=3.3; MW=288.4 grams/mole) were compared with those of the dyes listed in Table 1. Sudan I (Log(P)=5.5; MW=248.3 grams/mole) was selected as a model compound for testosterone based on a comparison of these parameters and commercial availability.

Saturated solutions of Sudan I in each of the excipients alpha-terpineol, tetraglycol, isostearic acid and propylene glycol were prepared (4 solutions) in screw cap vials by combining Sudan I with each excipient in separate vials and agitating the vials overnight at room temperature, then filtering the solutions to remove solid particulates. Saturated solutions of testosterone in each of the excipients alpha-terpineol, tetraglycol, isostearic acid and propylene glycol (4 solutions) were similarly prepared. Diffusion of the compounds through hairless mouse skin into phosphate buffer in a Franz cell was carried out and measured as described in Example 1, with Sudan I being used in place of Fat Brown RR.

A comparison of the cumulative amount of Sudan I and testosterone that were delivered across the hairless mouse skin into the buffer in the receiver chamber of the Franz cell as a function of time, expressed as micrograms of compound per ten milliliters of buffer solution (μg/10 mL), is reported in Table 3 (below).

TABLE 3

| | | CUMULATIVE AMOUNT OF COMPOUND | | | | | | |
|---|---|---|---|---|---|---|---|---|
| | | DIFFUSED ACROSS SKIN, μg/10 mL | | | | | | |
| | | | 6 | 12 | 18 | 24 | 50 | 59 |
| EXCIPIENT | COMPOUND | 0 hours | hours | hours | hours | hours | hours | hours |
| Isostearic | Sudan I | 0 | nm | nm | nm | 38 | 118 | 149 |
| acid | Testosterone | 0 | 74 | 192 | 418 | 740 | nm | nm |
| Propylene | Sudan I | 0 | nm | nm | nm | 31 | 108 | 142 |
| glycol | Testosterone | 0 | 79 | 216 | 546 | 956 | nm | nm |
| Tetraglycol | Sudan I | 0 | nm | nm | nm | 11 | 61 | 81 |
| | Testosterone | 0 | 23 | 46 | 81 | 119 | nm | nm |
| alpha- | Sudan I | 0 | nm | nm | nm | 72 | 201 | 259 |
| Terpineol | Testosterone | 0 | 605 | 1585 | 3043 | 4414 | nm | nm |

The results in Table 3 show that the same relative order (alpha-terpineol>tetraglycol) for membrane diffusion rate was obtained using testosterone and Sudan I, but different relative orders were obtained for propylene glycol and isostearic acid.

### Example 3

15 The molecular weight and log(P) values of levonorgestrel (Log(P)=3.48; MW=312.45 grams/mole) were compared with those of the dyes listed in Table 1. Disperse Red I (Log(P)=4.2; MW=314.35 grams/mole) was selected as a model compound for levonorgestrel based on a comparison of these parameters, as well as commercial availability and price.

Saturated solutions of Disperse Red 1 in each of the excipients alpha-terpineol, tetraglycol, isostearic acid and propylene glycol were prepared (4 solutions) in screw cap vials by combining Disperse Red 1 with each excipient in separate vials and agitating the vials overnight at room temperature, then filtering the solutions to remove solid particulates. Saturated solutions of levonorgestrel in each of the excipients alpha-terpineol, tetraglycol, isostearic acid and propylene glycol (4 solutions) were similarly prepared. Membrane diffusion measurements were obtained according to the procedure of Example 1, except that a crosslinked poly(dimethylsiloxane) membrane (0.51 mm thick membrane prepared by casting and thermally curing a curable silicone rubber obtained under the trade designation “DOW SYLGARD SILICONE 184” from Dow Corning Corporation, Midland, Mich.) was used instead of hairless mouse skin.

A comparison of the cumulative amount of Disperse Red 1 and levonorgestrel that were delivered across the poly(dimethylsiloxane) membrane into the buffer in the receiver chamber of the Franz cell as a function of time, expressed as micrograms of compound per milliliter of buffer solution (μg/mL), is reported in Table 4 (below).

TABLE 4

| | | CUMULATIVE AMOUNT OF | | | |
|---|---|---|---|---|---|
| | | COMPOUND DIFFUSED | | | |
| | | ACROSS SKIN, μg/10 mL | | | |
| | | 0 | 24 | 48 | 72 |
| EXCIPIENT | COMPOUND | hours | hours | hours | hours |
| Isostearic | Disperse Red 1 | 0 | 3 | 6 | 12 |
| acid | levonorgestrel | 0 | 5 | 9 | 12 |
| Propylene | Disperse Red 1 | 0 | 1 | 4 | 10 |
| glycol | levonorgestrel | 0 | 4 | 7 | 10 |
| Tetraglycol | Disperse Red 1 | 0 | 1 | 3 | 6 |
| | levonorgestrel | 0 | 4 | 8 | 10 |
| alpha- | Disperse Red 1 | 0 | 11 | 25 | 45 |
| Terpineol | levonorgestrel | 0 | 13 | 26 | 41 |

The results in Table 4 show that the same relative order (alpha-terpineol>isostearic acid>tetraglycol) for membrane diffusion rate was obtained using levonorgestrel and Disperse Red 1.

Various modifications and alterations of this invention will become apparent to those skilled in the art without departing from the scope and spirit of this invention, and it should be understood that this invention is not to be unduly limited to the illustrated embodiments set forth herein.

1. A method of formulating a pharmaceutical composition comprising:
comparing parameters of at least one pharmaceutical and a plurality of compounds, wherein the parameters comprise at least log(P) and molecular weight;
choosing at least one model compound from the plurality of compounds for each pharmaceutical;
providing at least one model compound-excipient formulation comprising at least one model compound and at least one excipient;
measuring the diffusion of a model compound of at least one model compound-excipient formulation across at least one membrane;
choosing a model compound-excipient formulation based on the measured model compound diffusion; and
combining components comprising the at least one pharmaceutical and the excipient package of the chosen model compound-excipient formulation.

2. A method according to claim 1, wherein the model compound-excipient formulation is saturated in model compound.

3. A method according to claim 1, wherein the parameters further comprise the number of freely rotatable bonds.

4. A method according to claim 1, wherein the parameters further comprise the number of H-bond donors and acceptors.

5. A method according to claim 1, wherein the diffusion is measured utilizing a Franz cell.

6. A method according to claim 1, wherein at least one model compound comprises a dye.

7. A method according to claim 6, wherein measuring the diffusion of the model compound comprises fluorescence spectroscopy.

8. A method according to claim 6, wherein the diffusion of the model compound is simultaneously measured in a plurality of diffusion cells.

9. A method according to claim 8, wherein measuring the diffusion of the model compound comprises recording an image.

10. A method according to claim 1, wherein at least one model compound-excipient formulation comprises a plurality of different excipients.

11. A method according to claim 1, wherein diffusion is measured utilizing a chemical reaction.

12. A method according to claim 1, wherein at least one membrane comprises a synthetic polymer membrane.

13. A method according to claim 1, wherein at least one membrane comprises skin.

14. A method according to claim 1, wherein at least one membrane is selected from the group consisting of hairless mouse skin, snake skin, pig skin, and cadaver skin.

15. A method according to claim 1, wherein the parameters consist of log(P) and molecular weight.

16. A method according to claim 1, wherein at least one parameter of at least one model compound is calculated.

17. A method according to claim 1, wherein at least one parameter of at least one model compound is experimentally determined.

18. A method according to claim 1, wherein at least one parameter of the pharmaceutical is calculated.

19. A method according to claim 1, wherein at least one parameter of the pharmaceutical is experimentally determined.

20. A method according to claim 1, further comprising:
contacting the pharmaceutical composition with the skin of a live mammal; and
observing the result.

21. A method according to claim 1, further comprising incorporating the pharmaceutical composition into a transdermal delivery system.

22. A method according to claim 21, further comprising contacting the pharmaceutical composition with the skin of a live mammal and observing the result.

23. A method according to claim 21, wherein the transdermal delivery device comprises an adhesive patch.

24. A method according to claim 1, wherein prior to measuring diffusion of each model compound-excipient formulation, it is incorporated into an adhesive patch.

25. A method according to claim 1, wherein the model compound-excipient formulation comprises a plurality of model compounds.
